(19) United States
(12) Patent Application Publication
(10) Pub. No.: US 2026/0250584 A1
(43) Pub. Date: Aug. 27, 2026

(54) SYSTEMS AND PROCESS FOR THE PRODUCTION OF HYDROCARBON PRODUCTS FROM CRUDE OIL

(71) Applicant: SABIC GLOBAL TECHNOLOGIES B.V., BERGEN OP ZOOM (NL)

(72) Inventors: Kenneth Francis LAWSON, Bangalore (IN); Mohammad Basheer AHMED, Bangalore (IN); Abdulrahman Shahid SARANG, Bangalore (IN); Ahmad M. AL-SHEHRI, Bangalore (IN); Hatem Abdallah BELFADHEL, Bangalore (IN)

(73) Assignee: SABIC GLOBAL TECHNOLOGIES B.V., BERGEN OP ZOOM (NL)

(21) Appl. No.: 18/879,204

(22) PCT Filed: Jul. 6, 2023
(86) PCT No.: PCT/EP2023/068777

§ 371 Date: Dec. 26, 2024

(30) Foreign Application Priority Data
Jul. 9, 2022 (IN) 202241039491

(51) Int. Cl. C10G 69/06; C10G 69/10; C10G 69/12

(52) U.S. Cl. CPC C10G 69/06; C10G 69/10; C10G 69/123; C10G 2300/1033; C10G 2400/20; C10G 2400/28

(57) ABSTRACT

Systems and processes to produce hydrocarbon products are described. A system can include a heavy hydrocarbon processing unit, a steam cracking, and a gaseous hydrocarbon separation unit coupled to the crude oil processing unit and the steam cracking unit. The gaseous hydrocarbon separation unit can separate naphtha, C4 hydrocarbons, and C2-C3 hydrocarbons from a gaseous hydrocarbon composition having a boiling temperature of less the 200° C. produced by the heavy hydrocarbon processing unit. The naphtha and C4 hydrocarbons can be stored. The C2-C3 hydrocarbons can be processed in the steam cracking unit to produce petroleum hydrocarbon products. The steam cracking unit can be indirectly coupled to an alkylation unit, a methyl t-butyl ether production unit, and/or a butene hydrogenation unit.

### CROSS REFERENCE TO RELATED APPLICATIONS

None.

### BACKGROUND OF THE INVENTION

### A. Field of the Invention

The invention generally concerns production of hydrocarbon products from crude oil, waste pyoil, or a blend thereof. A system can include a heavy hydrocarbon processing unit, a steam cracking, and a gaseous hydrocarbon separation unit coupled to the crude oil processing unit and the steam cracking unit. Naphtha, C4 hydrocarbons, and gaseous hydrocarbons can be separated in the gaseous hydrocarbon separation unit. The naphtha and/or C4 hydrocarbons can be stored. The gaseous hydrocarbons can be processed in the steam cracking unit. The steam cracking unit can be indirectly coupled to an alkylation unit, a methyl t-butyl ether production unit, and/or a butene hydrogenation unit.

### B. Description of Related Art

Systems and processes to produce hydrocarbon products have been described. For example, U.S. Pat. No. 11,180,706 to Al-Sayed et al. describes a configuration for olefins production. The processes progressively separate a crude oil into light and heavy fractions, which can be upgraded using fixed bed hydroconversion unit, a fluidized catalytic conversion unit, or a residue hydrocracking unit. The upgraded fluids can be fed to a steam cracking unit for production of olefins. This process suffers from less than optimal performance, which can increase capital cost of the overall process and lower profitability.

Overall, while the technologies of producing olefins exist, they can be energy inefficient and expensive.

### SUMMARY OF THE INVENTION

A discovery has been made that provides a solution to at least one of the problems associated with production of hydrocarbon products. In one aspect, the discovery can include a system that provides a gas separation unit coupled to a steam cracking unit and a hydrocarbon processing unit that includes an integrated separation unit and a hot hydrogen stripper. The integrated separation unit allows crude oil, a waste pyrolysis oil from a cracking unit (pyoil) feed stream, or a blend thereof to be processed into petroleum products. This configuration allows for the advantages of 1) lower cracking temperatures in the steam cracking unit and 2) increased ethylene selectivity. Without wishing to be bound by theory, it is believed that limiting the amount of naphtha and C4 hydrocarbons in the steam cracking unit, lowers the vapor pressure in the unit thus allowing C4+ hydrocarbons to be converted to ethylene at higher conversion and selectivity. Any C4 hydrocarbons produced from the steam cracking unit can be further processed to produce methyl t-butyl ether (MTBE) and/or alkylates. 2-Butenes produced in the production of MTBE can be isomerized to 1-butenes and residual alkenes. The residual alkenes can be hydrogenated to produce C4 hydrocarbons that can be provided to the steam cracking unit to continue the process. The system and processes of the present invention can provide a more cost and/or energy efficient means to produce petroleum products.

In one aspect of the present invention, systems and processes to produce hydrocarbon products are described. One system can include a heavy hydrocarbons processing unit, a gaseous hydrocarbon separation unit, a steam cracking unit, and at least one storage unit. The heavy hydrocarbons processing unit can be capable of producing a gaseous hydrocarbons that includes hydrocarbons having a boiling point less than 200° C., preferably less than 180° C., gas oil, and resid from a hydrocarbon composition that can include crude oil, a waste pyoil, or a blend thereof. The crude oil processing unit can include at least one integrated separation unit (IDS) and a hot hydrogen stripper (HHS). The gaseous hydrocarbon separation unit can be capable of receiving the gaseous hydrocarbons produced from the crude oil processing unit and can be capable of producing a naphtha stream, a gaseous stream comprising C4 hydrocarbons, and a C2-C3 hydrocarbon stream. The steam cracking unit can be capable of receiving the C2-C3 hydrocarbon unit and cracking the C2-C3 hydrocarbon stream to produce ethylene and propylene. A butane storage unit for storing the C4 hydrocarbons produced from the gaseous hydrocarbon separation unit and/or a naphtha storage unit for storing the naphtha produced from the gaseous hydrocarbon separation unit. The system can also include a MTBE production unit coupled to the steam cracking unit. The MTBE production unit can receive the C4 hydrocarbons from the cracking unit and produce 2-butenes and MTBE. The 2-butenes can be isomerized in a butene isomerization unit to produce a composition rich in 1-butenes and a residual butenes comprising 2-butenes. A butene hydrogenation unit can be coupled to the steam cracking unit and the butene isomerization unit. The butene hydrogenation unit can be capable of producing butane from butenes and providing the butanes to the steam cracking unit. In some aspects, the system includes an alkylation unit indirectly coupled to the steam cracking unit (e.g., a selective hydrogenation unit can be positioned downstream from the steam cracking unit and upstream from the alkylation unit). The alkylation unit can be capable of producing alkylates from the C4 hydrocarbons produced from the steam cracking unit and/or selective hydrogenation unit. In some aspects, the system can include a resid hydrocracking unit capable of receiving the heavy stream and configured to producing naphtha from the heavy hydrocarbon stream. The naphtha can be provided the steam cracking unit via a naphtha conduit. In some aspects, the system includes a hydrocracking unit capable of receiving the gas oil and configured to producing naphtha and unconverted oil (UCO) from the gas oil. The produced naphtha can be provided to the steam cracking unit via a second naphtha conduit. UCO is different from hydrocracking feed as it has been converted by hydro-treatment and includes saturated hydrocarbons.

Processes to produce hydrocarbon products using the systems of the present invention are also described. In one aspect, a process for the production of hydrocarbons can include (a) subjecting a hydrocarbon feed that includes crude oil, pyoil, or a blend thereof to conditions sufficient to produce a gaseous hydrocarbon stream that can include hydrocarbons have a boiling point less than 200° C., preferably 180° C., a gas oil stream, and a heavy hydrocarbon stream. Conditions in step (a) can include a temperature of 200 ° C. to 490° C. In step (b) the gaseous hydrocarbons can be separated into C2-C3 hydrocarbons, C4 hydrocarbons, and naphtha in the gaseous hydrocarbon separation unit. The gaseous hydrocarbons of step (a) can have a sulfur content of to less than 500 ppm, preferably less than 300 ppm. In some aspects, the gaseous hydrocarbons have a sulfur content less than 500 ppm prior to entering the gaseous hydrocarbon separation unit. The C4 hydrocarbons (e.g., butane) can be stored in a storage unit. Naphtha can also be stored in a storage unit. In step (c), the C2-C3 hydrocarbons can be subjected to steam cracking conditions sufficient to produce ethylene. In one aspect, the process can include subjecting the gas oil to conditions sufficient to produce a second naphtha composition and UCO. The second naphtha composition, the UCO, or a blend thereof can be provided to the steam cracking unit of step (c) to continue the process. In some aspects, the heavy hydrocarbons in step (a) can be subjected to conditions sufficient to produce a third naphtha stream and pitch. The third naphtha stream can be provided to the steam cracking unit of step (c) to further the process of producing hydrocarbon products. In the steam cracking unit, the second and third naphtha streams, the UCO stream, or a combination thereof, can be subjected to conditions sufficient to produce a second C4 hydrocarbon composition and ethylene. In some aspects, a portion of the second C4 hydrocarbon composition can be processed to produce MTBE. The MTBE process can produce a mixture of 1-butene and 2-butene, which can be subjected to conditions suitable to isomerize the 2-butenes to form a stream rich in 1-butenes and residual butenes containing unreacted 2-butenes. The unreacted 2-butenes can be hydrogenated to form butanes, which can be recycled to the steam cracking unit in step (c) to further the process. In some aspects, a portion of the second C4 hydrocarbon composition can be used to produce alkylates. The second and third naphtha streams, the UCO stream, or a combination thereof, can be subjected to conditions, in the steam cracking unit, sufficient to continue the process.

In another aspect, a process to produce hydrocarbon products can include (a) subjecting a C4 hydrocarbon composition comprising alkenes obtained from a steam cracking unit to conditions suitable to produce methyl t-butyl ether (MTBE) and alkenes comprising 2-butenes. In step (b) the alkenes of step (a) can be subjected to isomerization conditions suitable produce 1-butenes from the 2-butenes and residual butenes, and subjecting the 1-butenes to the step (a) process to produce additional MTBE. In step (c), the residual butenes of step (b) can be subjected to hydrogenation conditions suitable to produce alkanes comprising butanes, and the alkanes can be recycled to the steam cracking unit.

In yet another aspect of the present invention, a process to produce hydrocarbon products can include (a) subjecting a C4 hydrocarbon composition that can include alkynes, alkenes, or a mixture thereof obtained from a steam cracking unit to hydrogenation conditions suitable to selective hydrogenate the alkynes and alkenes and produce a composition comprising alkenes and alkanes. In step (b), the composition of step (a) can be subjected to conditions suitable to produce alkylates and hydrogenated C4 hydrocarbons. In step (c), at least a portion of the hydrogenated C4 hydrocarbons are recycled to the steam cracking unit.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification.

The term “C #hydrocarbons”, wherein “#” is a positive integer, is meant to describe all hydrocarbons having #carbon atoms. Moreover, the term “C #+ hydrocarbons” is meant to describe all hydrocarbon molecules having #or more carbon atoms. Accordingly, the term “C2+ hydrocarbons” is meant to describe a mixture of hydrocarbons having 2 or more carbon atoms. The term “C2+ alkanes” accordingly relates to alkanes having 2 or more carbon atoms.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The systems and processes of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the systems and processes of the present invention are their abilities to produce petrochemicals from crude oil.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 illustrates an embodiment of a system to produce hydrocarbon products from a crude feed that includes a crude oil processing unit coupled to a gaseous hydrocarbon separation unit, and a steam cracking unit coupled to the crude oil processing unit and the gaseous hydrocarbon separation unit.

FIG. 2 is an illustration of the system of FIG. 1 with additional processing units shown.

FIG. 3 illustrates an embodiment of the system of FIG. 1 that includes a butene isomerization unit coupled to a hydrogenation unit that is coupled to the steam cracking unit.

FIG. 4 illustrates an embodiment of the system of FIG. 1 that includes an alkylation unit indirectly coupled to the steam cracking unit.

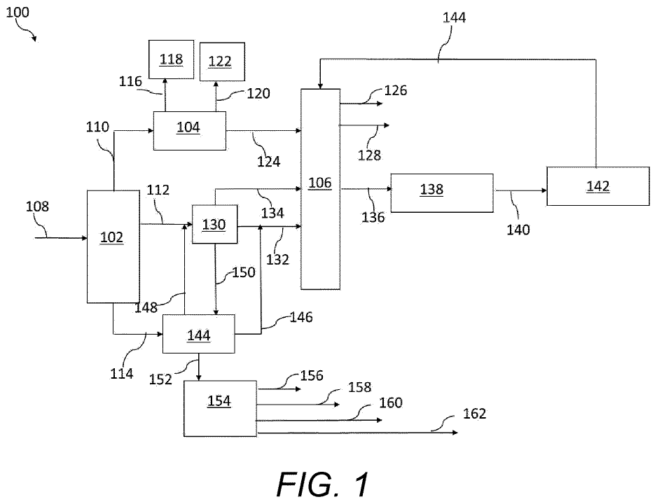

While the invention is susceptible to various modifications and alternative forms, specific embodiments thereof are shown by way of example in the drawings. The drawings may not be to scale. Still further, the schematics illustrated in FIGS. 1-4 can be combined with one another, which can be used, for example, to create a more robust process of producing a variety of petrochemical products from crude oil.

### DETAILED DESCRIPTION OF THE INVENTION

A discovery has been made that provides a solution to at least one of the problems associated with producing hydrocarbon products from a hydrocarbon composition that can include crude oil, pyoil from a cracking unit, or a blend thereof. In one aspect, the hydrocarbon composition can be processed in a heavy hydrocarbon processing unit to produce gaseous hydrocarbons having a boiling temperature of less than 200° C., preferably less than 180° C., gas oil, and heavy hydrocarbons. The gas oil can be provided to the steam cracking unit for further processing. By removing the hydrocarbons having a boiling point of less than 200° C. from one of the streams being fed to the steam cracking unit, lower cracking temperatures in the steam cracking unit and/or increased olefin (e.g., ethylene or propene) selectivity can be realized. Without wishing to be bound by theory, it is believed that limiting the amount of naphtha and/or C4 hydrocarbons in the steam cracking unit can lower the vapor pressure in the unit thus allowing C4+ hydrocarbons to be cracked to ethylene and propene at lower temperatures. Any C4 hydrocarbons produced in the steam cracking unit can be further processed in an MTBE processing unit to produce MTBE and/or an alkylation unit to produce alkylates. Providing a portion of the C4 hydrocarbons to an alkylation unit can reduce costs associated with MTBE production by decreasing the amount of methanol purchased for the MTBE process, while producing petrochemical products such as alkylates. Butenes generated in the MTBE process can be separated into 1-butenes and 2-butenes using isomerization methods. The 2-butene can be further hydrogenated to produce additional C4 hydrocarbons, which can be recycled to the steam cracking unit, to continue the process of the present invention. Overall, the process of the present invention can provide a more cost and/or energy efficient system for producing a variety of petrochemicals from crude oil. These and other non-limiting aspects of the present invention are discussed in further detail in the following sections with reference to the Figures.

FIG. 1 illustrates a system that produces petroleum products from crude oil, waste pyoil, or a blend thereof. Referring to FIG. 1, system 100 to produce petroleum products is described. System 100 can include a crude oil processing unit 102, a gaseous hydrocarbon separation unit 104, and a steam cracking unit 106. Crude oil can be the petroleum extracted from geologic formations in its unrefined form. The term crude oil can also include petroleum that has been subjected to water-oil separations and/or gas-oil separation and/or desalting and/or stabilization. Non-limiting examples of crude oil include Arabian Heavy, Arabian Light, other Gulf crudes, Brent, North Sea crudes, North and West African crudes, Indonesian, Chinese crudes, West Texas crude, and mixtures thereof, but also shale oil, tar sands, gas condensates and bio-based oils. The crude oil used as feed to the process of the present invention preferably is conventional petroleum having an API gravity of more than 20° API as measured by the ASTM D287 standard. In one aspect, the crude oil used in the process of the present invention is a light crude oil having an API gravity of more than 30° API. In another aspect, the crude oil used in the process of the present invention can include Arabian Light Crude Oil. Arabian Light Crude Oil typically has an API gravity of between 32-36° API and a sulfur content of between 1.5-4.5 wt. %. Pyoil can include waste fuel oil type material, with high aromatics and other multi-carbon ring structured molecules and optional elemental carbon.

Crude hydrocarbon stream 108 can include, crude oil, waste pyoil, or a mixture thereof and can enter heavy hydrocarbon processing unit 102. While one stream shown in FIG. 1, it is understood that crude oil and waste pyoil could enter heavy hydrocarbon processing unit via separate conduits. In heavy hydrocarbon processing unit 102, crude hydrocarbon stream 108 can be processed into a light hydrocarbon stream 110, gas oil hydrocarbon stream 112, and a resid stream 114. Light hydrocarbon stream can have a boiling temperature of less than 200° C., preferably less than 180° C. (e.g., 200° C. 195° C., 190° C., 180° C., 175° C. or less, or any range or value there between). Gas oil can be have a boiling point range of about 340-560° C., more preferably of about 350-550° C., more preferably 250-360° C., most preferably of about 260-350° C. Resid stream 114 can have a boiling point of more than about 490° C. Heavy hydrocarbon processing unit 102 can include a flash vessel, an integrated separation unit and a hot hydrogen stripper. Integrated separation units are known in the art and one skilled in the crude oil processing can determine the type of integrated separation unit required for the process. In the integrated separation unit, an initial separation of a low boiling fraction can be performed based on a combination of centrifugal and cyclonic effects to separate the light hydrocarbons having a boiling temperature less than 200° C. Having a fraction having a boiling temperature less than 200° C. can provide vapor control of the naphtha being processed in the steam cracking unit 106, thus increasing the conversion of higher boiling hydrocarbons to ethylene.

The hydrocarbons having a boiling temperature above 200° C. can be heated and pressurized using hot hydrogen to separate the resid hydrocarbons to produce a 200° C. to 490° C. hydrocarbon stream (gas oil). The hot hydrogen stripper can use utilize a hydrogen feed as the stripping medium and can be operated to provide broad flexibility, based on the composition of the crude hydrocarbon stream entering heavy hydrocarbon processing unit 102. The gas oil can be cooled, to recover hydrogen, and further processed as described herein. The recovered hydrogen can be fed to a downstream pressure swing adsorption (PSA) unit (not shown), after amine treatment (not shown), to improve the hydrogen purity. The PSA hydrogen product can be compressed in a make-up hydrogen compressor (not shown) to provide the make-up hydrogen for the one or more hydroprocessing units. In some embodiments, heavy hydrocarbon processing unit 102 can produce streams other than gas oil. By adjusting the amount of hydrogen fed to the hot hydrogen stripper, as well as the operating conditions of the hot hydrogen stripper and heater, the hydrocarbon cut points can be adjusted to produce other distillate (or UCO) fractions from the gas oil.

Light hydrocarbon stream 110 can exit crude oil processing unit 102 and enter gaseous hydrocarbon separation unit 104. In gaseous hydrocarbon separation unit 104, light hydrocarbon stream 110 can be separated into any one of any combination of or all of naphtha, C4 hydrocarbons, and/or C2-C3 hydrocarbons. Gaseous hydrocarbon separation unit can receive streams from other processing units (e.g., from a fixed bed hydrocracking unit, or a moving bed hydrocracking unit). The gaseous hydrocarbon separation unit can be coupled or be a part of a hydrocracking unit. Gaseous hydrocarbon separation unit 104 can include one or more distillation units known in the art capable of separating hydrocarbons having a boiling temperature of less than 200° C. For example, a naphtha stream at a boiling range of 20° C. to 190° C., a C4 hydrocarbon stream at −10° C. to 0 ° C., preferably −5° C. a C2/C3 hydrocarbon stream at −90 to −40° C. In some embodiments, gaseous hydrocarbon separation unit can include one or more units capable of removing sulfur containing compounds from light hydrocarbon stream 110. The sulfur level can be reduced to, or is, less than 500 ppm, preferably less than 300 ppm (e.g., 500, 450, 425, 400, 375, 350, 325, 300, 275, 250, 225, 200, 175, 150, 125, 100 ppm or less, or any range or value there between) using conventional methods. The light hydrocarbon can be processed (e.g., multiple distillations) to remove hydrogen and/or other impurities (e.g., CO₂/H₂S) and separated into naphtha, C4 hydrocarbons, and C2-C3 gases. Naphtha stream 116 can exit gaseous hydrocarbon separation unit 104 and enter storage unit 118. C4 hydrocarbon stream 120 can exit gaseous hydrocarbon separation unit 104 and enter storage unit 122.

C2-C3 hydrocarbon stream 124 can exit gaseous hydrocarbon separation unit 104 and enter steam cracking unit 106. In steam cracking unit 106, the C2-C3 feed can be subjected to steam cracking temperatures of 600° C. to 900° C. (e.g., 600° C., 625° C., 650° C., 675° C., 700° C., 725° C., 750° C., 775° C., 850° C., 875° C., 900° C., or any value or range there between) and/or a pressure of 0.2 MPa to 0.3 MPa (e.g., 0.2 MPa, 0.21 MPa, 0.22 MPa, 0.23 MPa, 0.24 MPa, 0.25 MPa, 0.26 MPa, 0.27 MPa, 0.28 MPa, 0.30 MPa, or any value or range there between). At such a temperature and pressure the ethane C2-C3 hydrocarbons are cracked to make ethylene and propylene. In a steam cracking process, the saturated hydrocarbons are broken down into smaller, often unsaturated, hydrocarbons such as ethylene and propylene by diluting the mixed hydrocarbon feed with steam and heating the mixture in a furnace in the absence presence of oxygen. The steam cracking reaction can have a residence times of 50-1000 milliseconds. Steam cracking unit can have a fractionation unit (not shown) or a gas fractionation unit (not shown) capable of separating alkanes from the olefinic products. Such fractionation units are well known in the art. Ethylene stream 126 can exit steam cracking unit 106, and be stored, sold, or used in other processing units. Propylene stream 128 can exit steam cracking unit 106, and be stored, sold, or used in other processing units.

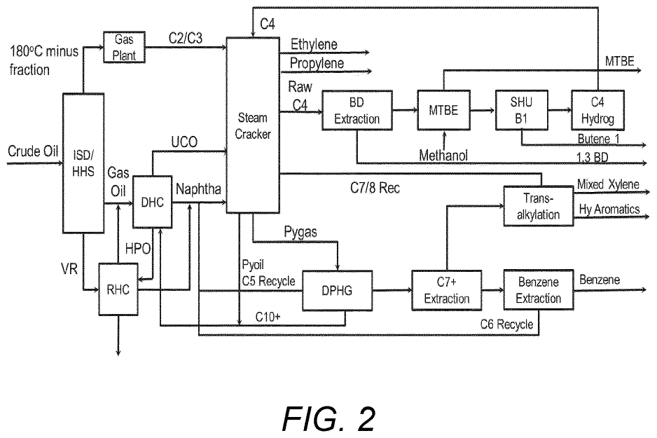

Gas oil stream 112 can exit crude oil processing unit 102 and enter gas oil processing unit 130. In gas oil processing unit 130, gas oil stream 112 can be subjected to a various distillations processes, hydrocracking process, or a combination thereof to produce a naphtha and UCO. Gas oil processing can include either a single fixed bed catalytic reactor or two such reactors in series together with one or more fractionation units to separate desired products from unconverted material and can also incorporate the ability to recycle unconverted material to one or both of the reactors. Gas oil processing reactors may be operated at a temperature of 200-600° C., preferably 300-400° C., a pressure of 3-35 MPa, preferably 5 to 20 MPa together with 5-20 wt. % of hydrogen (in relation to the hydrocarbon feedstock). Hydrogen can flow co-current with the hydrocarbon feedstock or counter current to the direction of flow of the hydrocarbon feedstock, in the presence of a dual functional catalyst active for both hydrogenation-dehydrogenation and ring cleavage, where aromatic ring saturation and ring cleavage may be performed. Catalysts used in such processes comprise one or more elements selected from the group consisting of Pd, Rh, Ru, Ir, Os, Cu, Co, Ni, Pt, Fe, Zn, Ga, In, Mo, W and V in metallic or metal sulfide form supported on an acidic solid such as alumina, silica, alumina-silica and zeolites. Naphtha stream 132 (second naphtha stream) and UCO stream 134 can each exit gas oil processing unit 130 and enter steam cracking unit 106 to continue the process to produce petroleum products (e.g., ethylene, propylene, and butane). FIG. 2 is an illustration of system 100, depicting one aspect of distillation, hydrocracking, hydrotreating units that can be used in gas oil processing unit 130.

In steam cracking unit 106, naphtha stream 132 and UCO stream 134 are subjected to steam cracking conditions previously described to produce ethylene, propylene, and C4 hydrocarbons, Pygas and C7/8 hydrocarbons. The C4 hydrocarbons can be a mixture of butadiene, butane, and butenes (e.g., ethyl acetylene, vinyl acetylene, 1,3-butadiene, 1,2-butadiene, isobutylene, cis-2-butene, trans-2-butene, 1-butene, isobutane, and n-butane).

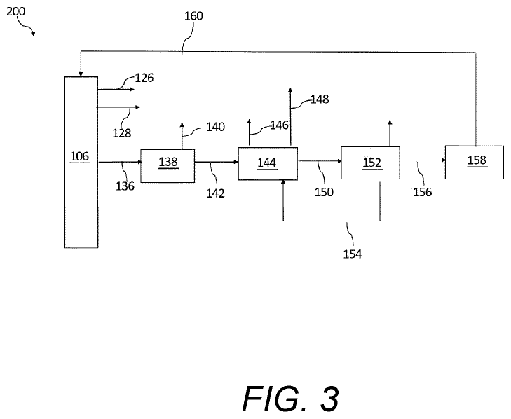

Referring to FIG. 3, in system 200, a portion or all of the C4 hydrocarbons produced from steam cracking unit 106 can be further processed produce methyl tert-butyl ether (MTBE) and additional C4 hydrocarbons. In some embodiment, the C4 hydrocarbons are produced from steam cracking unit 106 or different steam cracking units. C4 hydrocarbons stream 136 can exit steam cracking unit 106 and enter butadiene separation unit 138. In butadiene separation unit 138, butadiene can be separated from the C4 hydrocarbon to form a butadiene composition and a butene/butane composition. Butadiene stream 140 can exit butadiene separation unit 138 and be stored, transported or used in other processing units. Butene/butane stream 142 can exit butadiene separation unit 138 and enter methyl t-butyl ether (MTBE) production unit 140. In MTBE production unit 140, butene/butane stream 142 is contacted with methanol under conditions suitable to produce MTBE, a MTBE effluent, and 1-butene. MTBE stream 146 can exit MTBE production unit 140 and be stored and/or transported. 1-butene stream 148 can exit MTBE production unit 140 and be stored, used in other processing units and/or transported. MTBE effluent 150 can exit MTBE production unit 140 and enter butene isomerization unit 152. MTBE effluent can be a enriched 2-butene stream that includes 1-butene. In butene isomerization unit 152, 2-butene is contacted with a catalyst under isomerization conditions to produce a stream enriched in 1-butene and a residual butenes stream. The catalyst can by any known 2-butene isomerization catalyst. Isomerization conditions include reaction temperatures generally in the range of about 50° to 300° C. Reactor operating pressures usually can range from about atmospheric to 5 MPa. The amount of catalyst in the reactors can provide an overall weight hourly space velocity of from about 0.5 to 100 hr⁻¹. Enriched 1-butene stream 154 can exit butene isomerization unit 152 and enter MTBE production unit 140. Residual olefin stream 156 can exit butene isomerization unit and enter C4 alkenes/alkyne hydrogenation unit 158. In C4 alkenes/alkynes hydrogenation unit 158, C4 alkenes and alkyne can be contacted with a catalyst and hydrogen under conditions sufficient to produce additional C4 hydrocarbons.

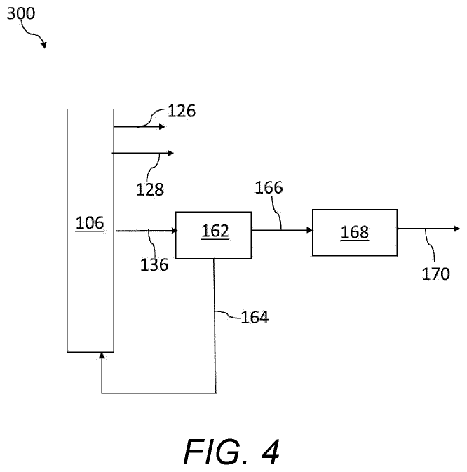

Referring to FIG. 4, in system 300, a portion or all of the C4 hydrocarbons produced from steam cracking unit 106 can be further processed to produce alkylates. In some embodiment, the C4 hydrocarbons are produced from steam cracking unit 106 or different steam cracking units. In system 300, a portion or all of C4 hydrocarbons stream 136 can exit steam cracking unit 106 and enter SHU (selective hydrogenation unit) 162. In SHU 162, the C4 hydrocarbons are subjected to conditions suitable to remove alkynes, dienes, and/or alkenes, and produce an alkene composition and additional C4 hydrocarbons. Additional C4 hydrocarbons 164 can exit SHU 162 and enter steam cracking unit 106 to continue the process. Alkene composition stream 166 can enter alkylation unit 168. Alkene composition can include alkenes and alkanes. In alkylation unit 168, the alkene composition is subjected to conditions to produce alkylates. Alkylate stream 170 can exit alkylation unit 168 and be stored, transported, or processed in other units. Any portion of or all portions of FIGS. 1 and 2 can be combined with any portion of or all portions of FIGS. 3 and/or 4, and vice versa.

Referring back to FIG. 1, resid stream 114 can exit crude oil processing unit 102 and enter resid hydrocracking unit 144. Resid hydrocracking unit 144 is capable of converting resid into naphtha and gas oil. Naphtha stream 146 (third naphtha stream) can exit reside hydrocracking unit 144 and be combined with naphtha stream 132 (second naphtha stream) or enter steam cracking unit 106 (not shown). Gas oil stream 148 can exit reside hydrocracking unit 144 and be combined with gas oil stream 112, or enter gas oil processing unit 130 to be further processed. Any resid hydrocracking processes can be used. For example, three basic reactor types can be employed in commercial hydrocracking which are a fixed bed (trickle bed) reactor type, an ebullated bed reactor type and slurry (entrained flow) reactor type. Fixed bed resid hydrocracking processes are capable of processing contaminated streams such as atmospheric residues and vacuum residues to produce the gas oil and naphtha. The catalysts used in fixed bed resid hydrocracking processes can include cobalt (CO), molybdenum (Mo), nickel (Ni), or a combination thereof on a refractory support, typically alumina. In case of highly contaminated feeds, the catalyst in fixed bed resid hydrocracking processes can also be replenished to a certain extend (moving bed). The process conditions can include a temperature of 350-450° C. and a pressure of 2-20 MPa gauge. Ebullated bed resid hydrocracking processes can continuously replace the catalyst, thus allowing the processing of highly contaminated feeds. The catalysts used in ebullated bed resid hydrocracking processes can include Co, Mo, Ni, or a combination thereof on a refractory support, typically alumina. The process conditions can include a temperature of 350-450° C. and a pressure of 5-25 MPa gauge. Slurry resid hydrocracking processes represent a combination of thermal cracking and catalytic hydrogenation to achieve high yields of distillable products from heavy resid feeds that are often highly contaminated. In a slurry reactor, the first liquid stage, thermal cracking and hydrocracking reactions can occur simultaneously in the bubble slurry phase at process conditions that include a temperature of 400-500 ° C. and a pressure of 15-25 MPa gauge. Resid, hydrogen and catalyst are introduced at the bottom of the reactor and a bubble slurry phase can be formed; the height of which depends on flow rate and desired conversion. In these processes, catalyst can be continuously replaced to achieve consistent conversion levels through an operating cycle. The catalyst can be an unsupported metal sulfide that is generated in situ within the reactor. The heavy-distillate produced by resid upgrading can be recycled to the resid hydrocracking unit 144 until extinction. In addition, higher boiling hydrocarbons (heavy pyrolysis oil (HPO) in FIG. 2) stream 150 can exit gas oil processing unit 130 and enter resid hydrocracking unit 144 to be further processed. Resid hydrocracking unit 144 can also produce pitch stream 152.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A system for the production of hydrocarbon products, the system comprising:
a heavy hydrocarbon processing unit capable of receiving crude oil, waste pyoil, or a mixture thereof and producing a gaseous hydrocarbon stream comprising hydrocarbons having a boiling point less than 200° C. and a gas oil stream;
a gaseous hydrocarbon unit capable of receiving the gaseous hydrocarbon stream comprising hydrocarbons having a boiling point less than 200° C. produced from the crude oil processing unit and capable of producing a naphtha stream, a gaseous stream comprising C4 hydrocarbons, a C2-C3 hydrocarbon stream.
a steam cracking unit capable of receiving the C2-C3 hydrocarbon stream and cracking the C2-C3 hydrocarbon stream to produce ethylene and propylene;
a storage unit coupled to the gaseous hydrocarbon unit and capable of receiving the gaseous stream comprising C4 hydrocarbons; and
a storage unit coupled to the gaseous hydrocarbon unit and capable of receiving the naphtha.

2. The system of any claim 1, wherein the crude oil processing unit comprises a integrated separation unit and a hot hydrogen separation unit.

3. The system of claim 1, further comprising:
a butene hydrogenation unit; and
a C4 conduit coupled to the steam cracking unit and the butene hydrogenation unit, the C4 conduit capable of providing C4 hydrocarbons from the butene hydrogenation unit to the steam cracking unit.

4. The system of claim 3, further comprising
a 2-butene isomerization unit coupled to the butene hydrogenation unit and capable of isomerizing 2-butene to 1-butene; and
a second C4 conduit coupled to the 2-butene isomerization unit and the butene hydrogenation unit, the C4 conduit capable of providing residual C4 alkenes from the 2-butene isomerization unit to the butene hydrogenation unit.

5. The system of claim 1, further comprising an alkylation unit positioned downstream to the steam cracking unit, the alkylation unit capable of receiving a stream comprising C4 alkenes and/or C4 alkanes and producing alkylates and optionally a selective hydrogenation unit positioned downstream from the steam cracking unit and upstream from the alkylation unit and capable of hydrogenating C4 alkenes and capable of recycling C4 alkenes and/or C4 alkanes to the steam cracking unit, and/or
further comprising:
a resid hydrocracking unit capable of receiving a resid stream produced by the heavy hydrocarbon processing unit and configured to produce naphtha from the resid stream; and
a naphtha conduit coupled to the resid hydrocracking unit and the steam cracking unit, the naphtha conduit capable of providing naphtha from the resid hydrocracking unit to the steam cracking unit, and/or further comprising:
a hydrocracking unit capable of receiving the gas oil and configured to producing naphtha and unconverted oil (UC) from the gas oil; and
a second naphtha conduit coupled to the hydrocracking unit and the steam cracking unit, the naphtha conduit capable of providing naphtha from the resid hydrocracking unit to the steam cracking unit.

6. A process for the production of hydrocarbons, the process comprising:
(a) subjecting a hydrocarbon composition comprising crude oil feed, waste pyoil, or a mixture thereof to conditions sufficient to produce gaseous hydrocarbons comprising hydrocarbons have a boiling point less than 200° C., gas oil, and heavy hydrocarbons;
(b) in a gaseous hydrocarbon unit, separating the gaseous hydrocarbons into C2-C3 hydrocarbons, C4 hydrocarbons, and naphtha;
(c) in a steam cracking unit, subjecting the C2-C3 hydrocarbons to conditions sufficient to produce ethylene, C4 hydrocarbons, and optionally propylene; and
(d) storing the C4 hydrocarbons and naphtha produced in step (b).

7. The process of claim 6, wherein the conditions in step (a) comprise a temperature of 200° C. to 490° C., and/or
further comprising subjecting the gas oil of step (a) to conditions sufficient to produce a second naphtha composition and unconverted oil (UCO), and/or
further comprising providing the second naphtha composition to the steam cracking unit of step (c), and/or
further comprising providing the UCO to the steam cracking unit of step (c).

8. The process of claim 7, further comprising subjecting the heavy hydrocarbons to conditions sufficient to produce a third naphtha composition.

9. The process of claim 8, further comprising providing the third naphtha composition to the steam cracking unit of step (c).

10. The process of claim 9, further comprising subjecting the second and third naphtha compositions, the UCO, or a combination thereof, in the steam cracking unit, to conditions sufficient to produce ethylene and a C4 hydrocarbon composition comprising alkenes.

11. The process of claim 10, wherein the C4 hydrocarbon composition is subjected, in a methyl t-butyl ether (MTBE) production unit, to conditions suitable to produce MTBE and alkenes comprising 2-butenes.

12. The process of claim 11, wherein the alkenes are subjected to isomerization conditions suitable produce 1-butenes from the 2-butenes and residual butenes, and providing the 1-butenes to the MTBE production unit.

13. The process of claim 12, wherein the residual butenes are subjected to hydrogenation conditions suitable to produce alkanes comprising butanes, and the alkanes are provided to the steam cracking unit of step (c).

14. The process of claim 8, wherein the gaseous hydrocarbons have a sulfur content of less than 500 ppm.

15. The process of claim 6, wherein C4 hydrocarbons produced from step (c) are hydrogenated and then are provided to an alkylation process, and optionally wherein at least a portion of the hydrogenated C4 hydrocarbons are recycled to the steam cracking unit.

16. A process to produce hydrocarbon products, the process comprising:
(a) subjecting a C4 hydrocarbon composition comprising alkenes obtained from a steam cracking unit to conditions suitable to produce methyl t-butyl ether (MTBE) and alkenes comprising 2-butenes;
(b) subjecting the alkenes of step (a) to isomerization conditions suitable produce 1-butenes from the 2-butenes and residual butenes, and subjecting the 1-butenes to the step (a) process to produce additional MTBE; and
(c) subjecting the residual butenes of step (b) to hydrogenation conditions suitable to produce alkanes comprising butanes, and providing the alkanes to the steam cracking unit.

17. A process to produce hydrocarbon products, the process comprising:
(a) subjecting a C4 hydrocarbon composition comprising alkynes obtained from a steam cracking unit to hydrogenation conditions suitable to selectively hydrogenate the alkynes and produce a composition comprising alkenes;
(b) subjecting the alkenes to conditions suitable to produce alkylates and hydrogenated C4 hydrocarbons; and
(c) recycling at least a portion of the hydrogenated C4 hydrocarbons to the steam cracking unit.
